(19) United States
(12) Patent Application Publication
(10) Pub. No.: US 2009/0054322 A1
(43) Pub. Date: Feb. 26, 2009

(54) Polypeptides and compositions comprising same and methods of using same for treating cxcr4 associated medical conditions

(75) Inventors: Nathan Karin, Haifa (IL); Gizi Wildbaum, Kiryat Yam (IL)

Correspondence Address: MARTIN D. MOYNIHAN d/b/a PRTSI, INC., P.O. BOX 16446, ARLINGTON, VA, 22215, US

(73) Assignee: Rappaport Family Institute for Research in the Medical Sciences, Haifa (IL)

(21) Appl. No.: 11/920,052

(22) PCT Filed: May 25, 2006
(86) PCT No.: PCT/IL2006/000626

§ 371 Date: Nov. 8, 2007

Related U.S. Application Data
(60) Provisional application No. 60/684,517, filed on May 26, 2005.

(51) Int. Cl. A61K 39/395; C07H 21/04; A61K 38/16; A61P 31/18; A61P 35/00; C07K 1/14

(52) U.S. Cl. 514/12; 536/23.1; 530/324; 530/350; 530/413; 424/130.1

(57) ABSTRACT

An isolated polynucleotide is provided, comprising a nucleic acid sequence encoding a soluble polypeptide which comprises an amino acid sequence of an N-terminus domain of CXCR4 and devoid of a CXCR4 extracellular domain selected from the group consisting of ECL1, ECL2 and ECL3, the soluble polypeptide being capable of binding SDF-1. Also provided are methods of using such a nucleic acid sequence such as for the treatment of cancer.

### FIELD AND BACKGROUND OF THE INVENTION

The present invention relates to novel polypeptides and compositions comprising same which can be used for treating CXCR4 associated medical conditions, such as cancer, inflammatory diseases, Type 1 Diabetes and HIV (AIDS).

Chemokines are small (˜8-14 kDa), cell secreted proteins, which, when activated, induce directed chemotaxis in nearby responsive cells. Members of this molecular superfamily share structural similarities, including four conserved cysteine residues that form disulphide bonds, crucial for protein structure and hence function. The location of the first two amino terminal cysteines in the protein sequence is used to classify chemokines into two main branches: the α-chemokines (also known as the CXC chemokines in which the cycteines are separated by a single amino acid), predominantly attracting and activating neutrophils and T lymphocytes and the β-chemokines (also known as the CC chemokines in which the cysteines are adjacent), affecting other blood cell types such as monocytes, lymphocytes, basophils, and eosinophils.

Chemokines bind to specific cell-surface receptors that belong to the G-protein-coupled seven-transmembrane-domain family, also termed “chemokine receptors”. Upon binding to their cognate ligands, chemokine receptors transduce an intracellular signal though the associated trimeric G proteins, resulting in, among other responses, a rapid increase in intracellular calcium concentration, changes in cell morphology, increased expression of cellular adhesion molecules, degranulation, and promotion of cell migration.

CXCR4 is a seven-transmembrane-spanning G-protein-coupled protein and a receptor for SDF-1/CXCL12 which is a CXC chemokine. The aforementioned factor is thought to be responsible for T cell trafficking and induction, for B-cell lymphopoiesis, bone marrow myelopoiesis and cardiac ventricular septum formation [Campbell, J. J., et al., Science, 279 381-383 (1998); Nagasawa, T. et al., Nature 382, 685-688 (1996)]. CXCR4 also functions as a co-receptor for T-cell-line-tropic HIV-1 [Feng, Y. et al., Science 272, 872-877 (1996)] and HIV-2 [Reeves et al., JVG 79: 1793-1799 (1998)]. CXCR4 has further been reported to be expressed in cultured endothelial cells [Volin, M. V. et al., Biochem. Biophys. Res. Commun. 242, 46-53 (1998)].

Insights into the physiological activity of SDF-1/CXCR4 interaction was provided by experiments showing genetically modified SDF-1 or CXCR4 knockout mice were embryonically lethal, expressing suppressed vascularization [US patent application 0040209837; Nagasawa, T. et al., Nature 382, 685-688 (1996)] In another study, lack of SDF-1 also resulted in reduction of B-cells and myeloid progenitors [Harihabu et al., J. Biol. Chem. 272:28726 (1997)]. These findings substantiate that the SDF-1/CXCR4 interaction is essential for B-cell lymphoesis, bone marrow myelopoiesis and neovascularization.

By using chimeric receptors composed of different domains from CXCR4 and CXCR2 (which does not bind SDF-1) domains, it was found that a chimera that lacks the CXCR4 distal N-terminal domain does not bind SDF-1 [Doranz et al, J. Virol. 73:2752 (1999)], although it is not clear whether lack of binding resulted from the alteration of the specific binding site, or because of a conformational change of the chimeric protein.

The SDF-1/CXCR4 interaction has a substantial affect on a wide range of cancer diseases. Numerous studies show that SDF-1 or CXCR4 are elevated in cancer patients or cell lines. For example, over expression of CXCR4 is associated with poor overall survival in nasopharyngeal carcinoma (NPC). High expression levels of SDF-1 are also correlated with tumor metastasis and reduced patient survival in patients with breast cancer [Kang H, et al., Breast Cancer Res.; 7(4):402-10 (2005)]. The CXCR4/SDF-1 pathway was also shown to be responsible for the site-specific predilection of breast cancer for bone. This, and data from other metastasis type cancers suggests an explanation for the preferential metastatic development of several cancerous diseases to specific areas, where SDF-1 concentration is high. For a review on the involvement of the SDF-1/CXCR4 interaction in the spread and progression of tumors, see Burger, J A and Kipps, T J, [Blood 1; 107(5):1761-7 (2006)].

Involvement of the SDF-1/CXCR4 interaction was shown for other malignant diseases including small cell lung cancer (SCLC), osteosarcoma, Inflammatory breast carcinoma (IBC) Giant cell tumor (GCT) of bone, acute myeloblastic leukemia (AML) and prostate cancer.

The interaction of SDF-1 with CXCR4 plays also a central role in the inflammatory process. For example, a vast majority of inflammatory cells in idiopathic inflammatory myopathies (IIM) were CXCR4 positive. A significant increase of SDF-1alpha and CXCR4 was observed in protein extracts of idiopathic inflammatory myopathies in comparison with normal controls [De Paepe B, et al., Neuromuscul Disord. 14(4):265-73 (2004)]. SDF-1 was also up-regulated in biliary epithelial cells (BEC) of inflammatory liver diseases such as viral hepatitis, liver cirrhosis, primary biliary cirrhosis, primary sclerosing cholangitis, and autoimmune hepatitis [Terada R, Lab Invest. 83 (5):665-72. (2003)]. Other inflammatory diseases characterized by high CXCR4 or SDF-1 expression are Atopic keratoconjunctivitis (AKC), diabetes [Kelly D J et al., Diab Vasc Dis Res 2(2):76-80 (2005)] and the associated diabetic retinopathy, and arthritis (U.S. Pat. No. 687,214). In multiple sclerosis it was speculated that SDF-1 might have a role in leucocyte extravasation, plasma cell persistence or axonal damage [Krumbholz M, et al., Brain. 129:200-11 (2006)], providing correlative evidence that SDF-1 antagonists could be useful therapeutics for this, and related diseases.

In addition, as mentioned hereinabove CXCR4 and CCR5 appear to be the principal coreceptors for HIV-1 in its natural cell entry mechanism [Zhang et al., J. Virol. 72:9337-9344 (1998)], depending on the viral strain and on the progression of the disease.

In view of the pivotal role of the SDF-1/CXCR4 interaction in cell migration and HIV infection, there is a need for antagonizing this receptor for the purpose of disease prevention and research.

Currently proposed antagonists for targeting the ligand (SDF-1) or the receptor (CXCR4) act on the protein or mRNA level, as summarized infra.

Use of an SDF derived peptide (T134) for the prevention of lung metastasis after injection of osteosarcoma cells in a mouse model, was taught by Perissinotto E, et al [Clin Cancer Res. 11:490-7 (2005)]. Limiting Lewis lung carcinoma, with a short SDF-1 peptide, serving as a CXCR4 antagonist in mice was also taught in U.S. Pat. No. 6,946,445. Another example is the CXCR4 antagonist AMD3100, proposed by AnorMED (British Columbia, Canada) to initiate stem cell mobilization for the purpose of stem cell accumulation for transplanting in cancer patients The same antagonist was found to reduce allergic lung inflammation in a mouse model [Lukacs N W, et al., Am J Pathol. 160(4):1353-60 (2002)].

Nucleic acid based agents such as siRNA which suppress CXCR4, were taught by Lapteva N, et al [Cancer Gene Ther. 12(1):84-9 (2005)] this treatment downregulated CXCR4 expression in human breast cancer cells, thereby decreasing breast cancer cell invasion and adhesion.

Use of an Anti SDF antibody (MAB 310; R&D Systems) was taught by Butler J M, et al. [Clin Invest. 115(1):86-93 (2005)] for the treatment of retinal neovascularization in a murine model of proliferative adult retinopathy. This anti SDF-1 antibody, trialed by RegenMed (Gainesville, Fla.), was shown to have a positive effect in treating adult retinopathy in primate, as well as mice models. Anti SDF-1 antibodies were used for the inhibiting migration of Giant cell tumor (GCT) of bone cells as well [Liao T S, et al., J Orthop Res. 23(1):203-9 (2005)].

Use of an anti CXCR4 antibody was suggested in U.S. Pat. No. 6,863,887 for the treatment of malignant diseases. A wide range of potential CXCR4 binding fragments of SDF-1 have been proposed for use in blocking HIV infection [see for example Feng, et al., Science 272:872 (1996) and Endres, et al., Cell 87:745 (1996); WO 9728258; WO 9804698]. But as these references also show, SDF-1 binding to CXCR4 does not depend on antagonism of the CXCR4 receptor. The second extracellular domain (ECL2) of CXCR4 seems to be the main domain that mediates interaction with the viral gp120 [Brelot E, et al., J. Virol. 73:2576-2586 (1999)]. Antibodies against this area were proposed for inhibiting the interaction with HIV, but use of various mAB against this area have shown that mAb recognizing overlapping epitopes have shown different inhibiting abilities to HIV-1 entrance [Carnec X, J. Virol.; 79(3): 1930-1933 (2005)]. This indicates that gp120 of any given isolate is able to recognize an array of CXCR4 conformations, and the use of an antibody against a specific epitope might not prove effective.

Collectively these data suggest that molecular strategies aimed at inhibiting the CXCR4/SDF-1 may prove useful in preventing the progression and metastasis of various cell migration and HIV viral infection associated medical conditions. However, a major problem associated with using antibodies to antagonize chemokine function is that they must be humanized before use in chronic human diseases. Another disadvantage in using antibodies is that their specificity on small epitopes might not inhibit conformation-dependent flexible interactions where a larger portion of the molecule is involved in the interaction.

Soluble receptor decoys have been previously suggested as therapeutic tools. Such molecules have been mostly utilized for mimicking single trans-membrane domain receptors. A major obstacle in applying the use of soluble receptor technology to chemokine receptors is that these receptors are G protein-coupled receptors that span the cell membrane seven times, and any attempt to generate their full length recombinant product results in a non-functional gene product.

U.S Pat. Appl. No. 20040132642 mentioned the use of soluble CXCR4 as an inhibitor of metastasis in a mammalian CXCR4 expressing tumor cell, but no description is provided as to the design of such molecules nor is experimental data regarding the use of such chimera is provided in this application.

U.S. Pat. Appl. No. 20040209837 teaches soluble CXCR4 as a therapeutic agent for suppressing vascularization, but this application illustrates the mortality of SDF-1 and CXCR4 lacking knock out mice, and does not provide any experimental or other data involving any utilization or outcome of the use of soluble CXCR4.

U.S. Pat. Appl. No. 20030091569 teaches soluble CXCR4 as a therapeutic agent for suppressing tumorigenesis. However this application illustrates the therapeutic inhibition of genes which are overexpressed in tumors, and suggests the use of a transmembrane domain deleted or inactivated form of CXCR4 for such a treatment, without providing any experimental or other data involving any utilization or outcome of the use of soluble CXCR4.

The abovementioned applications do not teach any applicable information for the production, utilization or results of using such soluble CXCR4.

There is thus a widely recognized need for, and it would be highly advantageous to have, compositions and methods using same for treating CXCR4 associated medical conditions which are devoid of the above limitations.

### SUMMARY OF THE INVENTION

According to one aspect of the present invention there is provided an isolated polynucleotide as set forth in SEQ ID NO: 1

According to another aspect of the present invention there is provided an isolated polynucleotide as set forth in SEQ ID NO: 3

According to yet another aspect of the present invention there is provided an isolated polynucleotide comprising a nucleic acid sequence encoding a soluble polypeptide which comprises an amino acid sequence of an N-terminus domain of CXCR4 and devoid of a CXCR4 extracellular domain selected from the group consisting of ECL1, ECL2 and ECL3, the soluble polypeptide being capable of binding SDF-1.

According to still another aspect of the present invention there is provided an isolated polynucleotide comprising a nucleic acid sequence encoding a soluble polypeptide which comprises an amino acid sequence of an ECL2 domain of CXCR4 and devoid of a CXCR4 extracellular domain selected from the group consisting of ECL1 and ECL3, the soluble polypeptide being capable of binding HIV.

According to an additional aspect of the present invention there is provided an isolated polypeptide comprising an amino acid sequence of an N-terminus domain of CXCR4 and devoid of a CXCR4 extracellular domain selected from the group consisting of ECL1, ECL2 and ECL3, the polypeptide being soluble and capable of binding SDF-1.

According to yet an additional aspect of the present invention there is provided an isolated polypeptide comprising an amino acid sequence of an ECL2 domain of CXCR4 and devoid of a CXCR4 extracellular domain selected from the group consisting of N-ter, ECL1 and ECL3, the polypeptide being soluble and capable of binding HIV.

According to still an additional aspect of the present invention there is provided an isolated polypeptide as set forth in SEQ ID NO: 2.

According to a further aspect of the present invention there is provided an isolated polypeptide as set forth in SEQ ID NO: 4.

According to yet a further aspect of the present invention there is provided the pharmaceutical composition comprising the isolated polypeptide and a pharmaceutically acceptable carrier or diluent.

According to still a further aspect of the present invention there is provided use of the isolated polypeptide for the manufacture of a pharmaceutical composition identified for treating a CXCR4 associated medical condition.

According to yet another aspect of the present invention there is provided a method of isolating SDF-1 from a biological sample, the method comprising: (a) contacting the biological sample with the isolated polypeptide, such that SDF-1 and the isolated polypeptide form a complex; and (b) isolating the complex to thereby isolate the SDF-1 from the biological sample.

According to further features in preferred embodiments of the invention described below, the polypeptide is as set forth in SEQ ID NO: 2, 4 or 6.

According to still further features in the described preferred embodiments the polypeptide further comprises a heterologous amino acid sequence conjugated to the amino acid sequence.

According to still further features in the described preferred embodiments the heterologous amino acid sequence comprises an immunoglobulin amino acid sequence.

According to still further features in the described preferred embodiments the heterologous amino acid sequence comprises a tag.

According to still further features in the described preferred embodiments the polypeptide is attached to a solid support.

According to still further features in the described preferred embodiments the polypeptide is attached to a non-proteinaceous moiety.

According to still further features in the described preferred embodiments the non-proteinaceous moiety is selected from the group consisting of polyethylene glycol (PEG), Polyvinyl pyrrolidone (PVP), poly(styrene comaleic anhydride) (SMA), and divinyl ether and maleic anhydride copolymer (DIVEMA).

According to still further features in the described preferred embodiments the pharmaceutically acceptable carrier is formulated for parenteral administration.

According to still further features in the described preferred embodiments the pharmaceutically acceptable carrier comprises a lipoamine acid.

According to still further features in the described preferred embodiments the pharmaceutically acceptable carrier comprises a carbohydrate.

According to still further features in the described preferred embodiments the pharmaceutically acceptable carrier comprises a microsphere.

According to still further features in the described preferred embodiments the pharmaceutically acceptable carrier comprises a liposome.

According to still further features in the described preferred embodiments the pharmaceutically acceptable carrier comprises a polymer microsphere.

According to still further features in the described preferred embodiments the isolated polypeptide being non-immunogenic.

According to still further features in the described preferred embodiments the CXCR4 associated medical condition is cancer or cancer metastasis.

According to still further features in the described preferred embodiments the CXCR4 associated medical condition is an inflammatory disease.

According to still further features in the described preferred embodiments the CXCR4 associated medical condition is AIDS.

According to still further features in the described preferred embodiments the CXCR4 associated medical condition is selected from the group consisting of idiopathic inflammatory myopathies (IIM), viral hepatitis, liver cirrhosis, primary biliary cirrhosis, primary sclerosing cholangitis, autoimmune hepatitis, cancer, cancer metastasis, small cell lung cancer (SCLC), osteosarcoma, inflammatory breast carcinoma (IBC), giant cell tumor (GCT) of bone, acute myeloblastic leukemia (AML), prostate cancer, multiple sclerosis, monocytic leukemia, arthritis, Atopic keratoconjunctivitis (AKC), diabetes and diabetic retinopathy.

The present invention successfully addresses the shortcomings of the presently known configurations by providing polypeptides and compositions which comprise the same for treating CXCR4-associated medical conditions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration depicting a nucleic acid construct encoding the human Ig-CXCR4 peptide of the present invention.

FIG. 2 is a bar graph depicting inhibition of SDF induced cell migration by CXCR4-Ig, as determined in a TransWell migration assay. THP-1 cells were reacted with SDF-1, CXCR4-Ig, a combination of SDF-1 and CXCr4-Ig, SDF-1 together with an anti-SDF-1 Ab (SDF+anti SDF), or medium (−; control cells). Following a three hour incubation, cell migration into the lower chamber of the transwell migration system was determined by FACS analysis. Note, presence of CXCR4-Ig inhibited SDF-induced cell migration in a similar manner as antibody mediated inhibition.

FIGS. 3a-b are graphs depicting the results of two independent experiments showing the inhibitory effect of the CXCR4-Ig on tumor metastasis to bone tissue as determined by a luciferase assay.

### DESCRIPTION OF THE PREFERRED EMBODIMENTS

The present invention is of isolated polynucleotides, polypeptides encoded therefrom, compositions comprising same and methods of using same for treating CXCR4 associated medical conditions, such as cancer and AIDS.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The chemokine receptor CXCR4 is a seven-transmembrane-spanning G-protein-coupled receptor which is capable of eliciting cellular signaling (e.g., increase in intracellular calcium) and resultant cellular responses (e.g., cell migration) upon SDF-1 binding thereto. CXCR4 is also a co-receptor participating in HIV-1 gp120 binding to mammalian host cells. Indeed CXCR4/SDF-1 interactions were found to play a central role in the pathogenicity mammalian (e.g., human) diseases, such as inflammatory diseases and cancer and as such were suggested as targets for drug development.

Soluble receptor decoys have been previously suggested as therapeutic tools. Such molecules have been mostly utilized for mimicking single trans-membrane domain receptors. A major obstacle in applying the use of soluble receptor technology to chemokine receptors is that the latter span the cell membrane seven times and any attempt to generate their full length recombinant product results in a non-functional gene product.

U.S. Pat. Appl. No. 20040132642 mentions the use of a soluble CXCR4 as an inhibitor of metastasis in a mammalian CXCR4 expressing tumor cell, but no description is provided as to the design of such molecules nor is experimental data regarding the use of such a chimera is provided in this application.

U.S. Pat. Appl. No. 20040209837 mentions soluble CXCR4 as a therapeutic agent for suppressing vascularization, but this application illustrates the mortality of SDF-1 and CXCR4 lacking knock out mice, and does not provide any experimental or other data involving any utilization or outcome of the use of soluble CXCR4.

U.S. Pat. Appl. No. 20030091569 mentions soluble CXCR4 as a therapeutic agent for suppressing tumorigenesis. These applications do not teach any applicable information for the production of a functional soluble CXCR4 nor do they teach utilization or results of using such a soluble CXCR4.

While reducing the present invention to practice, the present inventors have surprisingly uncovered that a chimeric molecule consisting of an immunoglobulin domain attached only to the N-terminus domain of CXCR4 is sufficient to inhibit SDF-1 binding to CXCR4 and to inhibit SDF-1 induced cell migration suggesting the use of such molecules in the treatment of CXCR4-associated medical conditions, such as cancer.

As shown in the Examples section which follows, the present inventors were able to inhibit tumor metastasis to bone tissue in a prostate cancer model using such a fusion polypeptide, substantiating its use as an anti cancer treatment.

Based on these findings the present invention also envisages the use of another CXCR4 polypeptide (e.g., ECL2 and optionally N-ter) for inhibiting HIV binding to host cells thus combating AIDS and related complications.

Thus, according to one aspect of the present invention there is provided an isolated soluble polypeptide which comprises an amino acid sequence of an N-terminus domain of CXCR4 and devoid of a CXCR4 extracellular domain selected from the group consisting of ECL1, ECL2 and ECL3 (e.g., either or all), said soluble polypeptide being capable of binding SDF-1. Examples of such polypeptides are as set forth in SEQ ID NO: 2 and 4. Such a polypeptide may be used as a decoy (dominant negative) by way of binding to SDF-1 and sequestering same from binding to cell-surface CXCR4.

According to another aspect of the present invention there is provided an isolated soluble polypeptide which comprises an amino acid sequence of an ECL2 domain of CXCR4 and optionally an N-ter domain of same and devoid of a CXCR4 extracellular domain selected from the group consisting of ECL1 and ECL3, said soluble polypeptide being capable of binding HIV-1. An example of such a polypeptide is as set forth in SEQ ID NO: 6.

The significance of ECL2 and optionally N-ter was derived from the following studies. Doranz et al [J. Virol 73(4): 2752-2761 (1999)] have found that replacing a few residues in the ECL2 domain alone in a chimeral CXCR4, was sufficient for diminishing viral entry. In addition, sequence differences between human and murine CXCR4 ECL2s were found to be responsible for murine CXCR4 lack of co-receptor activity [Parolin et al., Journal of Virology 72, 1652-1656 (1998)]. In another study using a rat/human CXCR4 chimera, it was shown that the HIV-1NDK isolate requires both the Nt and ECL2 for efficient fusion and entry, whereas HIV-1LAI only requires the presence of the CXCR4 ECL2 [Brelot et al., Journal of Virology 71, 4744-4751 (1997)]. HIV-2ROD also requires both the CXCR4Nt and ECL2 for fusion and entry [Reeves et al., JVG 79: 1793-1799 (1998)]. It is clear from the above, that ECL2 and, and optionally ECL2 can be used to inhibit HIV viral entry through CXCR4. Thus, polypeptides of this aspect of the present invention may be used as a decoy (dominant negative) by way of binding to HIV (e.g., HIV-1 and HIV-2) gp120 and sequestering same from binding to cell-surface CXCR4.

As used herein the term “CXCR4” refers to wild type CXCR4 or a variant thereof, such as but not limited to a mutant CXCR4, or a splice variants of CXCR4. Examples of CXCR4 proteins are as set forth in the following GenBank Accession Nos. XP_{—}541020, XP_{—}515811, NP_{—}071541, NP_{—}034041 and NP_{—}989948. Preferably CXCR4 is human CXCR4 such as provided in the following GenBank Accession Nos. NP_{—}001008540 and NP_{—}003458 or AY728138 (SEQ ID NO: 14).

As used herein “CXCR4 amino acid sequence” refers to a peptide portion of a mammalian (e.g., human) CXCR4 protein having affinity binding for SDF-1, HIV1 gp120 or HIV-2. CXCR4 N-ter domain corresponds to amino acid coordinates 1-39 (SEQ ID NO: 2) of GenBank Accession No. NP 003458; CXCR4 ECL-1 domain corresponds to amino acid coordinates 97-110 (SEQ ID NO: 7) of GenBank Accession No. NP_{—}003458; CXCR4 ECL-2 domain corresponds to amino acid coordinates 175-205 (SEQ ID NO: 6) of GenBank Accession No. NP_{—}003458; and CXCR4 ECL-3 domain corresponds to amino acid coordinates 263-282 (SEQ ID NO: 8) of GenBank Accession No. NP_{—}003458.

CXCR4 amino acid sequences of the present invention may be portions of above-listed naturally occurring sequences, active portions of same (i.e., capable of ligand binding e.g., SDF-1 binding), or mimetics of same as long as solubility is retained [essentially the protein lacks transmembrane domain(s) and therefore is secreted]. For example, amino acid residues at positions 7, 12 and 21 in the N terminus, as well as Aspartic acid at position 193 in ECL2 were found important for mediating pg 120 binding.

As used herein the term “mimetics” when made in reference to peptides refers to molecular structures, which serve as substitutes for the peptides of the present invention in interaction with for example SDF-1 [Morgan et al. (1989) Ann. Reports Med. Chem. 24:243-252 for a review of peptide mimetics].

Peptide mimetics, as used herein, include synthetic structures (known and yet unknown), which may or may not contain amino acids and/or peptide bonds, but retain the structural and functional features of a peptide ligand. Types of amino acids which can be utilized to generate mimetics are further described hereinbelow. The term, “peptide mimetics” also includes peptoids and oligopeptoids, which are peptides or oligomers of N-substituted amino acids [Simon et al. (1972) Proc. Natl. Acad. Sci. USA 89:9367-9371]. Further included as peptide mimetics are peptide libraries, which are collections of peptides designed to be of a given amino acid length and representing all conceivable sequences of amino acids corresponding thereto. Methods for the production of peptide mimetics are described hereinbelow.

Preferably the polypeptides of the present invention or compositions which comprise the same are designed non-immunogenic.

As used herein the term “non-immunogenic” refers to a substance which is substantially incapable of producing an immune response in a subject administered therewith. For example, non-immunogenic in a human means that upon contacting the polypeptide of this aspect of the present invention with the appropriate tissue of a human, no state of sensitivity or resistance to the polypeptide is demonstrable upon the second administration of that polypeptide after an appropriate latent period (e.g., 8 to 14 days).

It should be noted that a single CXCR4 amino acid sequence (e.g., N-ter, ECL-2) or an active portion thereof may be included in the polypeptides of the present invention, but inclusion of at least two CXCR4 amino acid sequences, each being capable of binding SDF-1, HIV-1 or HIV-2 (preferably with high affinity) may be preferred. Due to increased avidity, these polypeptides may be used as potent inhibitors and lower dosages may be administered.

As used herein “affinity binding” refers to a minimal K_{D} value of at least 10⁻⁶ M.

The term “peptide” as used herein encompasses native peptides (either degradation products, synthetically synthesized peptides or recombinant peptides) and as mentioned hereinabove, peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH₂—S, CH₂—S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc). For example, the N-ter domain of naturally occurring CXCR4 is typically sulfated, probably on tyrosine residues (sulfotyrosines). Inhibition of cellular sulfation pathways, including tyrosine sulfation, blocks CXCR4-mediated HIV-1 entry. It is therefore suggested that inclusion of such modification in the polypeptides of the present invention may improve polypeptide efficacy.

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1

| | Three-Letter | One-letter |
|---|---|---|
| Amino Acid | Abbreviation | Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchex | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchex | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| | Nnbhm | N-(N-(3,3-diphenylpropyl) | |
| N-(N-(2,2-diphenylethyl) | | carbamylmethyl(1)glycine | Nnbhe |
| carbamylmethyl-glycine | Nnbhm | | |
| 1-carboxy-1-(2,2-diphenyl | Nmbc | | |
| ethylamino)cyclopropane | | | |

Since the present peptides are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

Generation of peptide mimetics, as described hereinabove, can be effected using various approaches, including, for example, display techniques.

Thus, the present invention contemplates a display library comprising a plurality of display vehicles (such as phages, viruses or bacteria) each displaying at least 2, at least 3, at least 5, at least 7, at least 11, at least 15 consecutive amino acids derived from polypeptide sequences of the N-ter of CXCR4 (e.g., SEQ ID NO: 2).

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

Peptide mimetics can also be uncovered using computational biology. Software programs useful for displaying three-dimensional structural models, such as RIBBONS (Carson, M., 1997. Methods in Enzymology 277, 25), O (Jones, T A. et al., 1991. Acta Crystallogr. A47, 110), DINO (DINO: Visualizing Structural Biology (2001) http://www.dino3d.org); and QUANTA, INSIGHT, SYBYL, MACROMODE, ICM, MOLMOL, RASMOL and GRASP (reviewed in Kraulis, J., 1991. Appl Crystallogr. 24, 946) can be utilized to model interactions between SDF-1 and prospective peptide mimetics to thereby identify peptides which display the highest probability of binding to a specific SDF-1 region. Computational modeling of protein-peptide interactions has been successfully used in rational drug design, for further detail, see Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109, and Mauro M J. et al., 2002. J Clin Oncol. 20, 325-34.

Polypeptides of the present invention may further comprise at least one heterologous amino acid sequence conjugated to the CXCR4 amino acid sequence described hereinabove.

As used herein the phrase “heterologous amino acid sequence” refers to an amino acid sequence which does not form a part of the CXCR4 amino acid sequence. This sequence preferably confers solubility to the polypeptides of the present invention, preferably increasing the half-life of the chimeric molecule in the serum.

Such a heterologous amino acid sequence is generally localized at the amino- or carboxyl-terminus of the CXCR4 peptide of the present invention or at both ends.

For example, a CXCR4 amino acid sequence may be embedded between two heterologous sequences, such as described Hoogenboom (1991) Mol. Immunol. 28:1027-1037. The heterologous amino acid sequence may be attached to the CXCR4 amino acid sequence by any of peptide or non-peptide bond. Attachment of the CXCR4 amino acid sequence to the heterologous amino acid sequence may be effected by direct covalent bonding (peptide bond or a substituted peptide bond) or indirect binding such as by the use of a linker having functional groups. Functional groups include, without limitation, a free carboxylic acid (C(═O)OH), a free amino group (NH₂), an ester group (C(═O)OR, where R is alkyl, cycloalkyl or aryl), an acyl halide group (C(═O)A, where A is fluoride, chloride, bromide or iodide), a halide (fluoride, chloride, bromide or iodide), a hydroxyl group (OH), a thiol group (SH), a nitrile group (C—N), a free C-carbamic group (NR″—C(═O)—OR′, where each of R′ and R″ is independently hydrogen, alkyl, cycloalkyl or aryl).

An example of a heterologous amino acid sequence which may be used in accordance with this aspect of the present invention is an immunoglobulin sequence, such as the hinge and Fc regions of an immunoglobulin heavy domain (see U.S. Pat. No. 6,777,196). The immunoglobulin moiety in the chimeras of this aspect of the present invention may be obtained from IgG1, IgG2, IgG3 or IgG4 subtypes, IgA, IgE, IgD or IgM, as further discussed herein below.

Chimeras constructed from a receptor sequence linked to an appropriate immunoglobulin constant domain sequence (immunoadhesins) are known in the art. Immunoadhesins reported in the literature include fusions of the T cell receptor [Gascoigne et al., Proc. Natl. Acad. Sci. USA, 84: 2936-2940 (1987)]; CD4 [Capon et al., Nature 337: 525-531 (1989); Traunecker et al., Nature, 339: 68-70 (1989); Zettmeissl et al., DNA Cell Biol. USA, 9: 347-353 (1990); Byrn et al., Nature, 344: 667-670 (1990)]; L-selectin (homing receptor) [(Watson et al., J. Cell. Biol., 110:2221-2229 (1990); Watson et al., Nature, 349: 164-167 (1991)]; CD44 [Aruffo et al., Cell, 61: 1303-1313 (1990)]; CD28 and B7 (Linsley et al., J. Exp. Med., 173: 721-730 (1991)]; CTLA-4 [Lisley et al., J. Exp. Med. 174: 561-569 (1991)]; CD22 [Stamenkovic et al., Cell, 66:1133-1144 (1991)]; TNF receptor [Ashkenazi et al., Proc. Natl. Acad. Sci. USA, 88: 10535-10539 (1991); Lesslauer et al., Eur. J. Immunol., 27: 2883-2886 (1991); Peppel et al., J. Exp. Med., 174:1483-1489 (1991)]; NP receptors [Bennett et al., J. Biol. Chem. 266:23060-23067 (1991)]; and IgE receptor a [Ridgway et al., J. Cell. Biol., 115:abstr. 1448 (1991)].

Typically, in such fusions the chimeric molecule will retain at least functionally active hinge and CH2 and CH3 domains of the constant region of an immunoglobulin heavy chain. Fusions can also be generated to the C-terminus of the Fc portion of a constant domain, or immediately N-terminal to the CH1 of the heavy chain or the corresponding region of the light chain.

The exact site at which fusion (conjugation) between the heterologous sequence and the CXCR4 amino acid sequence is not critical. Particular sites are well known in the art and may be selected in order to optimize the biological activity, secretion or binding characteristics of the chimeric molecules of this aspect of the present invention.

Though it may be possible to conjugate the entire heavy chain constant region to the CXCR4 amino acid sequence of the present invention, it is preferable to fuse shorter sequences. For example, a sequence beginning in the hinge region just upstream of the papain cleavage site, which defines IgG Fc chemically; residue 216, taking the first residue of heavy chain constant region to be 114, or analogous sites of other immunoglobulins, is used in the fusion. In a particularly preferred embodiment, the CXCR4 amino acid sequence is fused to the hinge region and CH2 and CH3, or to the CH1, hinge, CH2 and CH3 domains of an IgG1, IgG2, or IgG3 heavy chain (see U.S. Pat. No. 6,777,196). The precise site at which the fusion is made is not critical, and the optimal site can be determined by routine experimentation.

As mentioned, the immunoglobulin sequences used in the construction of the chimeric molecules of this aspect of the present invention may be from an IgG immunoglobulin heavy chain constant domain. The use of human IgG1 and IgG3 immunoglobulin sequences is preferred. A major advantage of using IgG1 is that IgG1 can be purified efficiently on immobilized protein A. In contrast, purification of IgG3 requires protein G, a significantly less convenient medium. However, other structural and functional properties of immunoglobulins should be considered when choosing the Ig fusion partner for a particular chimera construction. For example, the IgG3 hinge is longer and more flexible, so it can accommodate larger CXCR4 amino acid sequences that may not fold or function properly when fused to IgG1. Another consideration may be valency; IgG are bivalent homodimers, whereas Ig subtypes like IgA and IgM may give rise to dimeric or pentameric structures, respectively, of the basic Ig homodimer unit. Other considerations in selecting the immunoglobulin portion of the chimeric molecules of this aspect of the present invention are described in U.S. Pat. No. 6,77,196.

Thus, polypeptides of the present invention may comprise a heterologous amino acid sequence, as described above and further described in the Examples section which follows (e.g., SEQ ID NO: 4).

Additionally or alternatively as mentioned hereinabove CXCR4 amino acid sequences of the present invention may be attached to a non-proteinaceous moiety, such molecules are selected non-immunogenic in a subject. Such a molecule is highly stable (resistant to in-vivo proteaolytic activity probably due to steric hindrance conferred by the non-preoteinaceous moiety) and may be produced using common solid phase synthesis methods which are inexpensive and highly efficient, as further described hereinbelow. However, it will be appreciated that recombinant techniques may still be used, whereby the recombinant peptide product is subjected to in-vitro modification (e.g., PEGylation as further described hereinbelow).

As mentioned, the CXCR4 amino acid sequence is attached to a non-proteinaceous moiety. The phrase “non-proteinaceous moiety” as used herein refers to a molecule not including amino acids (peptide bonded) that is attached to the above-described CXCR4 amino acid sequence. According to presently preferred embodiments the non-proteinaceous moiety of this aspect of the present invention is a polymer or a co-polymer (synthetic or natural). Non-limiting examples of the non-proteinaceous moiety of the present invention include polyethylene glycol (PEG), Polyvinyl pyrrolidone (PVP), divinyl ether and maleic anhydride copolymer (DIVEMA; see for example, Kaneda Y, et al., 1997, Biochem. Biophys. Res. Commun. 239: 160-5) and poly(styrene comaleic anhydride) (SMA; see for example, Mu Y, et al., 1999, Biochem Biophys Res Commun. 255: 75-9).

Bioconjugation of such a non-proteinaceous moiety confers the CXCR4 amino acid sequence with stability (e.g., against protease activities) and/or solubility (e.g., within a biological fluid such as blood, digestive fluid) while preserving its biological activity and prolonging its half-life. Bioconjugation is advantageous particularly in cases of therapeutic proteins which exhibit short half-life and rapid clearance from the blood. The increased half-lives of bioconjugated proteins in the plasma results from increased size of protein conjugates (which limits their glomerular filtration) and decreased proteolysis due to polymer steric hindrance. Generally, the more polymer chains attached per peptide, the greater the extension of half-life. However, measures are taken not to reduce the specific activity of the CXCR4 amino acid sequence of the present invention (i.e., ligand binding such as HIV binding or SDF-1 binding).

Bioconjugation of the CXCR4 amino acid sequence with PEG (i.e., PEGylation) can be effected using PEG derivatives such as N-hydroxysuccinimide (NHS) esters of PEG carboxylic acids, monomethoxyPEG₂-NHS, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldimidazol-activated PEGs, PEG-thiol, PEG-maleimide. Such PEG derivatives are commercially available at various molecular weights [See, e.g., Catalog, Polyethylene Glycol and Derivatives, 2000 (Shearwater Polymers, Inc., Huntsvlle, Ala.)]. If desired, many of the above derivatives are available in a monofunctional monomethoxyPEG (mPEG) form. In general, the PEG added to the CXCR4 amino acid sequence of the present invention should range from a molecular weight (MW) of several hundred Daltons to about 100 kDa (e.g., between 3-30 kDa). Larger MW PEG may be used, but may result in some loss of yield of PEGylated peptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher. PEGylation of molecules is further discussed in, e.g., Hermanson, Bioconjugate Techniques, Academic Press San Diego, Calif. (1996), at Chapter 15 and in Zalipsky et al., “Succinimidyl Carbonates of Polyethylene Glycol,” in Dunn and Ottenbrite, eds., Polymeric Drugs and Drug Delivery Systems, American Chemical Society, Washington, D.C. (1991).

Conveniently, PEG can be attached to a chosen position in the CXCR4 amino acid sequence by site-specific mutagenesis as long as the activity of the conjugate is retained (e.g., SDF-1 binding). For example, a Cysteine residue on the CXCR4 amino acid sequence as set forth in SEQ ID NO: 2 can be a target for PEGylation. Computational analysis may be effected to select a preferred position for mutagenesis without compromising the activity.

Various conjugation chemistry of activated PEG such as PEG-maleimide, PEG-vinylsulfone (VS), PEG-acrylate (AC), PEG-orthopyridyl disulfide can be employed. Methods of preparing activated PEG molecules are known in the arts. For example, PEG-VS can be prepared under argon by reacting a dichloromethane (DCM) solution of the PEG-OH with NaH and then with di-vinylsulfone (molar ratios: OH 1:NaH 5:divinyl sulfone 50, at 0.2 gram PEG/mL DCM). PEG-AC is made under argon by reacting a DCM solution of the PEG-OH with acryloyl chloride and triethylamine (molar ratios: OH 1:acryloyl chloride 1.5:triethylamine 2, at 0.2 gram PEG/mL DCM). Such chemical groups can be attached to linearized, 2-arm, 4-arm, or 8-arm PEG molecules.

While conjugation to cysteine residues is one convenient method by which the CXCR4 amino acid of the present invention can be PEGylated, other residues can also be used if desired (also those of the heterologous amino acid sequence). For example, acetic anhydride can be used to react with NH₂ and SH groups, but not COOH, S—S, or —SCH₃ groups, while hydrogen peroxide can be used to react with —SH and —SCH₃ groups, but not NH₂. Reactions can be conducted under conditions appropriate for conjugation to a desired residue in the peptide employing chemistries exploiting well-established reactivities.

For bioconjugation of the CXCR4 amino acid sequence of the present invention with PVP, the terminal COOH-bearing PVP is synthesized from N-vinyl-2-pyrrolidone by radical polymerization in dimethyl formamide with the aid of 4,4′-azobis-(4-cyanovaleric acid) as a radical initiator, and 3-mercaptopropionic acid as a chain transfer agent. Resultant PVPs with an average molecular weight of Mr 6,000 can be separated and purified by high-performance liquid chromatography and the terminal COOH group of synthetic PVP is activated by the N-hydroxysuccinimide/dicyclohexyl carbodiimide method. The CXCR4 amino acid sequence is reacted with a 60-fold molar excess of activated PVP and the reaction is stopped with amino caploic acid (5-fold molar excess against activated PVP), essentially as described in Haruhiko Kamada, et al., 2000, Cancer Research 60: 6416-6420, which is fully incorporated herein by reference.

Resultant conjugated CXCR4 molecules (e.g., PEGylated or PVP-conjugated CXCR4) are separated, purified and qualified using e.g., high-performance liquid chromatography (HPLC). In addition, purified conjugated molecules of this aspect of the present invention may be further qualified using e.g., in vitro assays in which the binding specificity of SDF-1 or HIV to its receptor (e.g., CXCR4) is tested in the presence or absence of the CXCR4 conjugates of the present invention, essentially as described for other chemokines [e.g., MIP-1α, see for example, Hesselgesser J, 1998 (Supra), which is fully incorporated herein by reference].

Polypeptides (conjugated or not) of this aspect of present invention can be biochemically synthesized such as by using standard solid phase techniques. These methods include exclusive solid phase synthesis, partial solid phase synthesis methods, fragment condensation and classical solution synthesis. These methods are preferably used when the peptide is relatively short (i.e., 10 kDa) and/or when it cannot be produced by recombinant techniques (i.e., not encoded by a nucleic acid sequence, such as a “Tag” further described hereinbelow) and therefore involve different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Briefly, an isolated (i.e., isolated from its natural environment) polynucleotide which comprises a nucleic acid sequence encoding a polypeptide of the present invention can be ligated into an expression construct (i.e., expression vector), of the present invention positioned under the transcriptional control of a regulatory element, such as a promoter, is introduced into host cells.

For example, a nucleic acid sequence encoding a CXCR4 peptide of the present invention (e.g., SEQ ID NO: 1) is ligated in frame to an immunoglobulin cDNA sequence so as to generate a chimeric fusion (e.g., SEQ ID NO: 3). It will be appreciated that, ligation of genomic immunoglobulin fragments can also be used. In this case, fusion requires the presence of immunoglobulin regulatory sequences for expression. cDNAs encoding IgG heavy-chain constant regions can be isolated based on published sequence from cDNA libraries derived from spleen or peripheral blood lymphocytes, by hybridization or by polymerase chain reaction (PCR) techniques. The nucleic acid sequences encoding the CXCR4 amino acid sequence and immunoglobulin can be ligated in tandem into an expression construct (vector) that directs efficient expression in the selected host cells, further described hereinbelow. For expression in mammalian cells, pRK5-based vectors [Schall et al., Cell, 61:361-370 (1990)]; and CDM8-based vectors [Seed, Nature, 329:840 (1989)] can be used. The exact junction can be created by removing the extra sequences between the designed junction codons using oligonucleotide-directed deletional mutagenesis [Zoller et al, Nucleic Acids Res., 10:6487 (1982); Capon et al., Nature, 337:525-531 (1989)]. Synthetic oligonucleotides can be used, in which each half is complementary to the sequence on either side of the desired junction; ideally, these are 11 to 48-mers. Alternatively, PCR techniques can be used to join the two parts of the molecule in-frame with an appropriate vector.

Methods of introducing the expression construct into a host cell are well known in the art and include, electroporation, lipofection and chemical transformation (e.g., calcium phosphate). See also Example 1 of the Examples section which follows.

The “transformed” cells are cultured under suitable conditions, which allow the expression of the chimeric molecule encoded by the nucleic acid sequence.

Following a predetermined time period, the expressed chimeric molecule is recovered from the cell or cell culture, and purification is effected according to the end use of the recombinant polypeptide.

Depending on the host/vector system utilized, any of a number of suitable transcription and translation elements including constitutive and inducible promoters, transcription enhancer elements, transcription terminators, and the like, can be used in the expression vector [see, e.g., Bitter et al., (1987) Methods in Enzymol. 153:516-544].

Other than containing the necessary elements for the transcription and translation of the inserted coding sequence (encoding the chimera), the expression construct of the present invention can also include sequences engineered to optimize stability, production, purification, yield or toxicity of the expressed fusion protein.

A variety of prokaryotic or eukaryotic cells can be used as host-expression systems to express the fusion protein coding sequence. These include, but are not limited to, microorganisms, such as bacteria transformed with a recombinant bacteriophage DNA, plasmid DNA or cosmid DNA expression vector containing the chimera coding sequence; yeast transformed with recombinant yeast expression vectors containing the chimera coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the chimera coding sequence. Mammalian expression systems are preferably used to express the chimera of the present invention.

The choice of host cell line for the expression of the molecules depends mainly on the expression vector. Eukaroyotic exoression systems are preferred (e.g., mammalian and insects) since they allow post translational modifications (e.g., glyccosylation). Another consideration is the amount of protein that is required. Milligram quantities often can be produced by transient transfections. For example, the adenovirus EIA-transformed 293 human embryonic kidney cell line can be transfected transiently with pRK5-based vectors by a modification of the calcium phosphate method to allow efficient expression. CDM8-based vectors can be used to transfect COS cells by the DEAE-dextran method (Aruffo et al., Cell, 61:1303-1313 (1990); Zettmeissl et al., DNA Cell Biol. US, 9:347-353 (1990)]. If larger amounts of protein are desired, the molecules can be expressed after stable transfection of a host cell line (see Example 1 of the Examples section). It will be appreciated that the presence of a hydrophobic leader sequence at the N-terminus of the molecule will ensure processing and secretion of the molecule by the transfected cells.

It will be appreciated that the use of bacterial or yeast host systems may be preferable to reduce cost of production. However since bacterial host systems are devoid of protein glycosylation mechanisms, a post production glycosylation may be needed.

In any case, transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant chimera molecule of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant protein is effected. The phrase “recovering the recombinant protein” refers to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Polypeptides of the present invention are preferably retrieved in “substantially pure” form. As used herein, “substantially pure” refers to a purity that allows for the effective use of the protein in the diverse applications, described hereinbelow.

Chimeric polypeptides comprising immunoglobulin amino acid sequence can be conveniently purified by affinity chromatography. The suitability of protein A as an affinity ligand depends on the species and isotype of the immunoglobulin Fc domain that is used in the chimera. Protein A can be used to purify chimeric molecules that are based on human γ1, γ2, or γ4 heavy chains [Lindmark et al., J. Immunol. Meth., 62:1-13 (1983)]. Protein G is preferably used for all mouse isotypes and for human γ3 [Guss et al., EMBO J., 5:1567-1575 (1986)]. The solid support to which the affinity ligand is attached is most often agarose, but other solid supports are also available. Mechanically stable solid supports such as controlled pore glass or poly(styrenedivinyl)benzene allow for faster flow rates and shorter processing times than can be achieved with agarose. The conditions for binding the chimeric molecules to the protein A or G affinity column are dictated entirely by the characteristics of the Fc domain; that is, its species and isotype. Generally, when the proper ligand is chosen, efficient binding occurs directly from unconditioned culture fluid. One distinguishing feature of chimeric molecules of this aspect of the present invention is that, for human .gamma.1 molecules, the binding capacity for protein A is somewhat diminished relative to an antibody of the same Fc type. Bound chimeric molecules of this aspect of the present invention can be efficiently eluted either at acidic pH (at or above 3.0), or in a neutral pH buffer containing a mildly chaotropic salt. This affinity chromatography step can result in an chimeric molecule preparation that is >95% pure. Medical grade purity is essential for therapeutic applications.

Other methods known in the art can be used in place of, or in addition to, affinity chromatography on protein A or G to purify chimeric molecules which include an immunoglobulin portion. Such chimeric molecules behave similarly to antibodies in thiophilic gel chromatography [Hutchens et al., Anal. Biochem., 159:217-226 (1986)] and immobilized metal chelate chromatography [Al-Mashikhi et al., J. Dairy Sci., 71:1756-1763 (1988)]. In contrast to antibodies, however, their behavior on ion exchange columns is dictated not only by their isoelectric points, but also by a charge dipole that may exist in the molecules due to their chimeric nature.

Polypeptides of the present invention may be used to treat CXCR4 associated medical conditions.

Thus, according to another aspect of the present invention, there is provided a method of treating CXCR4 associated medical condition in a subject in need thereof. The method comprising administering to the subject a therapeutically effective amount of the polypeptides (at least one) of the present invention, thereby treating the CXCR4 associated medical condition in the subject.

As used herein the term “subject” refers to a mammal, preferably a human subject (also encompassed are animals for veterinary purposes).

As used herein the term “treating” refers to preventing, curing, reversing, attenuating, alleviating, minimizing, suppressing or halting the deleterious effects of a CXCR4 associated medical condition.

As used herein the phrase “CXCR4 associated medical condition” refers to a disease, condition or disorder which depends on the interaction between CXCR4 ligand (HIV-1 pg 120, SDF-1) and its receptor, CXCR4, for onset or progression.

Examples of CXCR4 associated medical condition include, but are not limited to, inflammatory diseases, cancer, cancer metastasis and AIDS as well as related conditions.

A number of diseases and conditions, which involve an inflammatory response can be treated using the polypeptides described hereinabove. Examples of such diseases and conditions are summarized infra.

Inflammatory diseases—Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med. Sci. 2000 April; 319 (4):234), myasthenic diseases, Lambert-Eaton myasthenic syndrome (Takamori M. Am J Med. Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad. Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3): 178); antiphospholipid syndrome (Flamholz R. et al., J Clin Apheresis 1999; 14 (4):171); heart failure, agonist-like beta-adrenoceptor antibodies in heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDeviff H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Also envisaged is the treatment of, whim syndrome, also called warts, hypogammaglobulinemia, infections, and myelokathexis. WHIM syndrome is an immunodeficiency disease characterized by neutropenia, hypogammaglobulinemia and extensive human papillomavirus (HPV) infection. Despite the peripheral neutropenia, bone marrow aspirates from affected individuals contain abundant mature myeloid cells, a condition termed myelokathexis.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2): 157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type I diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med. Sci. 2000 April; 319 (4):234), myasthenia, Lambert-Eaton myasthenic syndrome (Takamori M. Am J Med. Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad. Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

The molecule of the present invention can be administered to the subject per se, or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism. Preferably, the pharmaceutical composition is not immunogenic.

As used herein, the term “active ingredient” refers to the molecule of the present invention accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Thus for example, the pharmaceutically acceptable carrier of the present invention may comprise a lipoamine acid.

Alternatively, the pharmaceutically acceptable carrier used by the present invention may comprise an embedding material such as a polyol (i.e., a carbohydrate). Non-limiting examples of carbohydrates which are suitable for use as excipients include maltodextrin (e.g., Glucidex Roquette), trehalose (e.g., Trehalose Merck), cellobiose, glucose, fructose, maltulose, iso-maltulose, lactulose, maltose, gentobiose, lactose, isomaltose, maltitol (e.g., Maltisorb Roquette), lactitol, erythritol, palatinitol, xylitol, mannitol, sorbitol, dulcitol and ribitol, sucrose, raffinose, gentianose, planteose, verbascose, stachyose, melezitose, dextran and inositol.

Yet alternatively, the pharmaceutically acceptable carrier used by the present is a microsphere suitable for oral administration. For example, the microsphere can include a water insoluble matrix of organic material that is resistant to dissolution or acidic degradation at pH levels found in the stomach (e.g., a pH level lower than 4) essentially as described in U.S. Pat. No. 6,849,271 to Vaghefi, et al., which is fully incorporated herein by reference. Such organic matrix material can be, for example, triglyceride, hydrogenated vegetable oil, a wax or a mixture of waxes, polyalkoxyalkylether, polyalkoxyalkylester and water insoluble partially degraded proteins.

It will be appreciated that the bioconjugated polymer (e.g., the PEGylated CXCR4 peptide of the present invention) can be used in, and as a part of, the pharmaceutically acceptable carrier, and thus serves as a carrier molecule for delivery of the CXCR4 amino acid sequence, while at the same time serving as a component of the delivery vehicle. A preferred embodiment of this dual use is a liposomal vehicle, e.g., PEG-conjugated liposomes, as described e.g., in U.S. Pat. Appl. No. 20030186869 to Poiani, George et al., which is fully incorporated herein by reference.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured to by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

It will be appreciated that the polypeptide (e.g., chimeric proteinicious) of this aspect of the present invention can be provided to the subject by means of gene therapy. Hence the above-described mammalian expression construct can be administered to the subject employing any suitable mode of administration, described hereinabove (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the subject (i.e., ex-vivo gene therapy).

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence such as the Igic leader sequence (e.g., SEQ ID NOs. 7 and 8). Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

The affinity of the CXCR4 peptide of the present invention its ligand allows use thereof in purification and detection of SDF-1 or HIV.

Thus, according to yet another aspect of the present invention there is provided a molecule comprising a tag and the CXCR4 polypeptide of the present invention.

As used herein the term “tag” refers to a moiety which is specifically recognized by a binding partner such as an antibody, a chelator or an avidin (biotin) molecule. The tag can be placed C-terminally or N-terminally of the CXCR4 peptide, as long as it does not interfere with a biological activity thereof (e.g., SDF-1 binding).

For example, a tag polypeptide has enough residues to provide an epitope (i.e., epitope tag) against which an antibody thereagainst can be made, yet is short enough such that it does not interfere with biological activity of the CXCR4 peptide. The epitope tag preferably also is fairly unique so that the antibody thereagainst does not substantially cross-react with other epitopes. Suitable tag polypeptides generally have at least six amino acid residues and usually between about 8-50 amino acid residues (preferably between about 9-30 residues). Preferred are poly-histidine sequences, which bind nickel, allowing isolation of the tagged protein by Ni-NTA chromatography as described (Lindsay et al. Neuron 17:571-574 (1996)], for example.

Such epitope-tagged forms of the CXCR4 are desirable, as the presence thereof can be detected using a labeled antibody against the tag polypeptide. Also, provision of the epitope tag enables the CXCR4 peptide of the present invention to be readily purified by affinity purification using the anti-tag antibody. Affinity purification techniques and diagnostic assays involving antibodies are described later herein.

Tag polypeptides and their respective antibodies are well known in the art. Examples include the flu HA tag polypeptide and its antibody 12CA5 (Field et al., Mol. Cell. Biol., 8:2159-2165 (1988)]; the c-myc tag and the 8F9, 3C7, 6E10, G4, B7 and 9E10 antibodies thereto (Evan et al., Molecular and Cellular Biology, 5:3610-3616 (1985)]; and the Herpes Simplex virus glycoprotein D (gD) tag and its antibody. Paborsky et al., Protein Engineering, 3(6):547-553 (1990). Other tag polypeptides have been disclosed. Examples include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)]. Once the tag polypeptide has been selected, an antibody thereto can be generated using methods which are well known in the art. Such antibodies are commercially available such as from Sigma, St. Louis. USA.

The molecules of the present invention can be used to isolate CXCR4 ligands from biological samples or to detect presence thereof (i.e., analyte) therein.

As used herein the phrase “biological sample” refers to a biological fluid such as blood, serum, plasma, lymph, bile fluid, urine, saliva, sputum, synovial fluid, semen, tears, cerebrospinal fluid, bronchioalveolar large fluid, ascites fluid, pus, conditioned medium and the like in which the analyte is present is present.

For example, isolation of SDF-1 may be effected as follows. First, contacting the biological sample with the polypeptide of the present invention, such that SDF-1 and the molecule form a complex (using buffer, temperature conditions which allow binding of the molecule to SDF-1); and isolating the complex to thereby isolate SDF-1 from the biological sample.

In order to isolate the complex, the molecule is preferably immobilized on a solid support. As used herein the phrase “solid support” refers to a non-aqueous matrix to which a reagent of interest (e.g., the molecule of this aspect of the present invention) can adhere. Examples of solid supports, include, but are not limited to, solid supports formed partially or entirely of glass (e.g., controlled pore glass), polysaccharides (e.g., agarose), polyacrylamides, polystyrene, polyvinyl alcohol and silicones. In certain embodiments, depending on the context, the solid support can comprise the well of an assay plate; in others it is a purification column (e.g., an affinity chromatography column). This term also includes a discontinuous solid phase of discrete particles, such as those described in U.S. Pat. No. 4,275,149.

Alternatively, such molecules can be used to detect presence of HIV in biological samples. For diagnostic applications, molecules typically will be labeled with a detectable moiety. The detectable moiety can be any one which is capable of producing, either directly or indirectly, a detectable signal. For example, the detectable moiety may be a radioisotope, a fluorescent or chemiluminescent compound, or a tag (such as described hereinabove and to which a labeled antibody can bind). The molecules of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

The molecules of this aspect of the present invention can be included in a diagnostic kit, in which the molecule and optionally solid support and imaging reagents (e.g., antibodies, chromogenic substrate etc.) can be packaged in suitable containers with appropriate buffers and preservatives and used for diagnosis.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Ig-CXCR4 and Stable Expression in Cell Lines Generation of CXCR4-Ig Construct

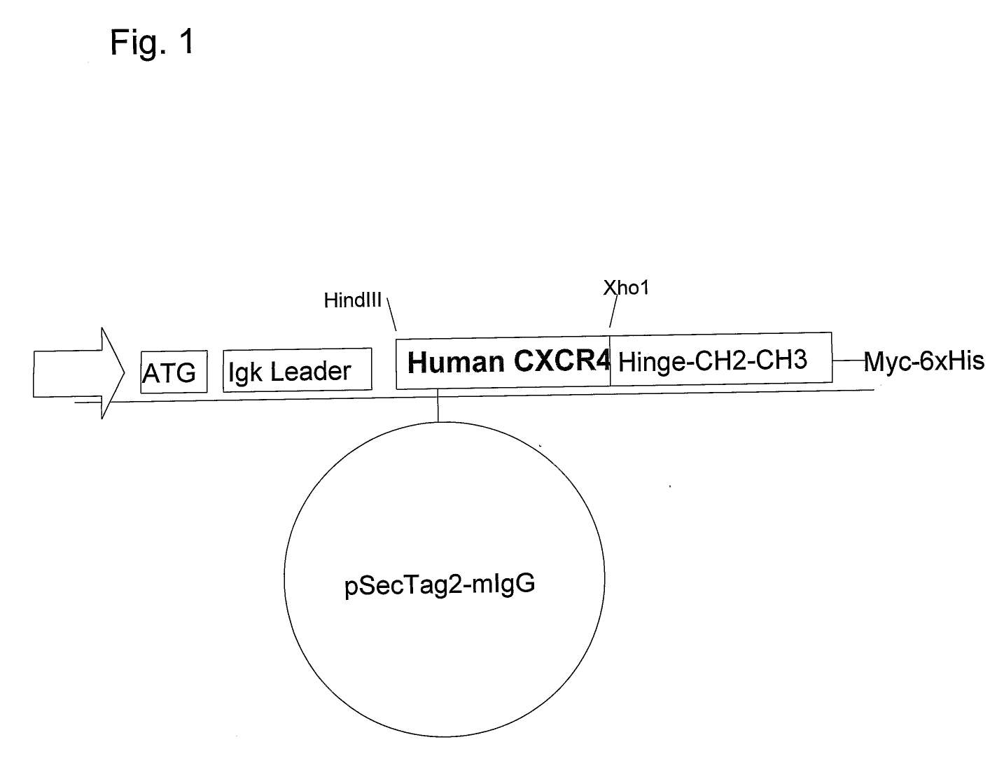

The N-terminal part (amino acids 1-39) of CXCR4 (GenBank Accession No. AY728138) is thought to participate in binding SDF-1 [Doranz, B. J., et al., J. Virol. 73:2752 (1999)]. Therefore in order to generate a soluble CXCR4 which may be used as a potent antagonist of SDF-1, a CXCR4 subdomian which includes the N terminus was in-frame fused to the Hinge-CH₂—CH₃ of human IgG1 heavy chain as schematically demonstrated in FIG. 1.

Materials and Methods

A cDNA encoding the constant region (Hinge-CH2—CH3) of human IgG1 heavy chain was cloned from LPS and IL-4 activated peripheral blood mononuclear cells onto pSecTag2/Hygro B (Invitrogen, San Diego, Calif.). The following primers were used for cloning the N terminus domain of CXCR4: sense-cccaagcttatggaggggatcagtatata and antisense-ccgctcgaggattttattgaaattagca (SEQ ID NOs. 11-12). The N-terminus was isolated from cDNA prepared from peripheral blood mononuclear cells and cloned in frame onto the pSecTag2/Hygro B plasmid containing the human Hinge-CH2—CH3. Following sequence verification the amplified PCR product (about 117 bp) was cloned into the pSecTag2 vector (Invitrogen, San Diego, Calif.). Hinge-CH2—CH3 of the human IgG Fcγ was ligated to the plasmid (pSec-CXCR4) down stream of the CXCR4 to create a fusion protein CXCR4-IgG.

Generation of Stable pSec-CXCR4-IgG-Expressing Cell Lines

The pSec-CXCR4-IgG plasmid was co-transfected into the DG44 CHO cells (DHFR4^{-/-}; ATCC accretion number: CRL-9096), with CHO DHFR minigene vector using jet PEI (Polypluse transfection—Illkirch Cedex, France) according the manufacturer's protocol. Stably transfected cells were selected in medium containing hygromycine (200 μg/ml). hCXCR4-IgG fusion protein was purified from the supernatants by a protein G-Sepharose column obtained from Amersham Biosciences (Uppsia, Sweden) and verified by western blot analysis using the goat anti human IgG-HRP (Sigma, St. Louis, Mo.).

### Example 2

### CXCR4-Ig Inhibits SDF-1-Induced Cell Migration

The ability of CXCR4-Ig to inhibit SDF-1 induced migration of THP-1 cells was tested using a TransWell chemotaxis assay.

Materials and Experimental Procedures

Cell Lines

THP-1 cells were obtained from American Type Culture Collection (ATCC, Rockville, Md. with ATCC Accession No. TIB-202) and grown according to the manufacturers protocol.

Antibodies—Anti SDF Ab was purchased from R&D Systems, Minneapolis, N. Mex., Biotest Catalog Number. MAB310.

Cell migration assay—Chemotaxis assays were conducted using a TransWell chamber (Corning Costar, Cambridge, Mass.). THP-1 cells with medium (1×10⁶ cells/well) were added to the inside chamber of the Transwell. After equilibration of the lower chambers with medium, chambers were supplemented with human recombinant SDF-1 (10 ng per well; R&D Systems, Minneapolis, N. Mex.), CXCR4-Ig (100 μg/ml), a combination of SDF-1 and CXCr4-Ig, SDF-1 together with an anti-SDF-1 Ab (SDF+anti SDF), or medium (−; control cells). Transwells were then incubated for 3 hours at 37° C. in humidified air containing 7.5% CO₂. Migrating monocytes were collected from the lower chamber and counted by FACS analysis.

Results

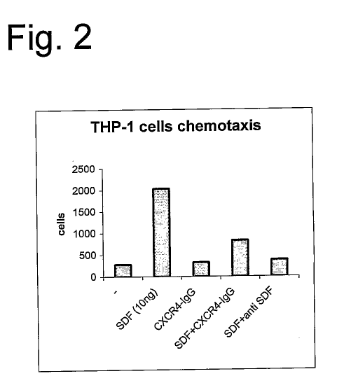

As shown in FIG. 2, addition of soluble CXCR4-Ig significantly and pronouncedly blocked SDF-1 induced cell migration (FIG. 2, p<0.001), FIG. 2 shows a pronounced reduction of the relative number of migrating cells, as compared to SDF induced migrating cells. Presence of CXCR4-Ig inhibited SDF-induced cell migration in a similar manner as antibody mediated inhibition. When SDF was inhibited, either by antibody or CXCR4-Ig, resulting cell migration was similar to non SDF induced, basal cell migration (control chambers).

### Example 3

### CXCR4-Ig Inhibits Tumor Metastasis

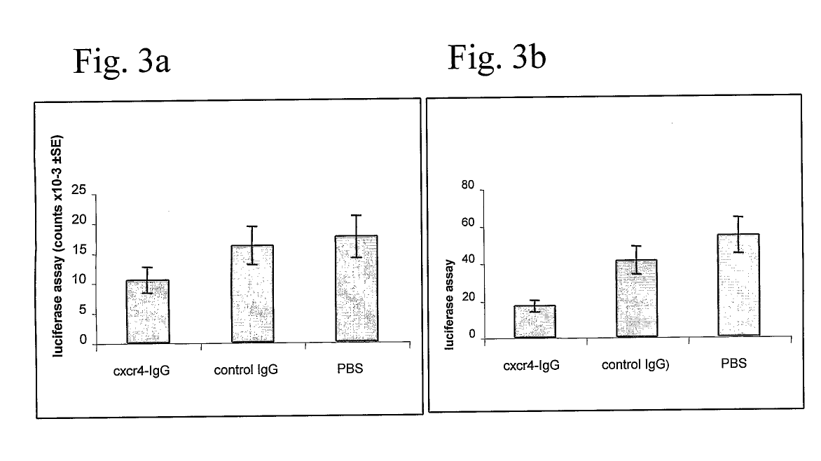

To determine the effect of the CXCR4-IgG fusion of the present invention on metastatic spread to the bone, groups of 6 SCID/Bg mice were inoculated with 5×10⁶ PC-3 cells transfected with the Luciferase gene (PC-3.luc) per mouse. On day 12 following implantation of the primary tumor mice were treated, twice a week, with either 200 μg of the CXCR4-Ig fusion protein, isotype matched control IgG, or PBS. 40 days later all mice were sacrificed, bones were harvested, homogenized in lysis buffer and the light emission from cell extract was detected using the luciferase 1000 assay system (Promega). A TD-20/20 Luminometer (Turner Designs, Inc., Sunnyvale, Calif.) determined light emission from cell extract. Results of 2 consecutive experiments (FIGS. 3a and b) are shown. As shown the chimeric molecule was able to inhibit tumor metastasis to the bone by at least 30% supporting its use as an anti cancer drug.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. An isolated polynucleotide as set forth in SEQ ID NO: 1.

2. An isolated polynucleotide as set forth in SEQ ID NO: 3.

3. An isolated polynucleotide comprising a nucleic acid sequence encoding a soluble polypeptide which comprises an amino acid sequence of an N-terminus domain of CXCR4 and devoid of a CXCR4 extracellular domain selected from the group consisting of ECL1, ECL2 and ECL3, said soluble polypeptide being capable of binding SDF-1.

4. An isolated polynucleotide comprising a nucleic acid sequence encoding a soluble polypeptide which comprises an amino acid sequence of an ECL2 domain of CXCR4 and devoid of a CXCR4 extracellular domain selected from the group consisting of ECL1 and ECL3, said soluble polypeptide being capable of binding HIV.

5. An isolated polypeptide comprising an amino acid sequence of an N-terminus domain of CXCR4 and devoid of a CXCR4 extracellular domain selected from the group consisting of ECL1, ECL2 and ECL3, said polypeptide being soluble and capable of binding SDF-1.

6. An isolated polypeptide comprising an amino acid sequence of an ECL2 domain of CXCR4 and devoid of a CXCR4 extracellular domain selected from the group consisting of N-ter, ECL1 and ECL3, said polypeptide being soluble and capable of binding HIV.

7. An isolated polypeptide as set forth in SEQ ID NO: 2.

8. An isolated polypeptide as set forth in SEQ ID NO: 4.

9. A pharmaceutical composition comprising the isolated polypeptide of claim 5, and a pharmaceutically acceptable carrier or diluent.

10. A method of treating a CXCR4 associated medical condition in a subject in need thereof, the method comprising administering to the subject a therapeutically effective amount of the polypeptide of claim 5, thereby treating the CXCR4 associated medical condition.

11. A method of isolating SDF-1 from a biological sample, the method comprising:
(a) contacting the biological sample with the isolated polypeptide of claim 5, such that SDF-1 and the isolated polypeptide form a complex; and
(b) isolating said complex to thereby isolate the SDF-1 from the biological sample.

12-25. (canceled)

26. The method of claim 10, wherein said CXCR4 associated medical condition is cancer or cancer metastasis.

27. The method of claim 10, wherein said CXCR4 associated medical condition is an inflammatory disease.

28. The method of claim 10, wherein said CXCR4 associated medical condition is AIDS.

29. The method of claim 10, wherein said CXCR4 associated medical condition is selected from the group consisting of idiopathic inflammatory myopathies (IIM), viral hepatitis, liver cirrhosis, primary biliary cirrhosis, primary sclerosing cholangitis, autoimmune hepatitis, cancer, cancer metastasis, small cell lung cancer (SCLC), osteosarcoma, inflammatory breast carcinoma (IBC), giant cell tumor (GCT) of bone, acute myeloblastic leukemia (AML), prostate cancer, multiple sclerosis, monocytic leukemia, arthritis, Atopic keratoconjunctivitis (AKC), diabetes and diabetic retinopathy.

30. A pharmaceutical composition comprising the isolated polypeptide of claim 6 and a pharmaceutically acceptable carrier or diluent.

31. A pharmaceutical composition comprising the isolated polypeptide of claim 7 and a pharmaceutically acceptable carrier or diluent.

32. A pharmaceutical composition comprising the isolated polypeptide of claim 8 and a pharmaceutically acceptable carrier or diluent.

33. A method of treating a CXCR4 associated medical condition, in a subject in need thereof, the method comprising administering to the subject a therapeutically effective amount of the polypeptide of claim 6, thereby treating the CXCR4 associated medical condition.

34. A method of treating a CXCR4 associated medical condition, in a subject in need thereof, the method comprising administering to the subject a therapeutically effective amount of the polypeptide of claim 7, thereby treating the CXCR4 associated medical condition.

35. A method of treating a CXCR4 associated medical condition, in a subject in need thereof, the method comprising administering to the subject a therapeutically effective amount of the polypeptide of claim 8, thereby treating the CXCR4 associated medical condition.

36. A method of isolating SDF-1 from a biological sample, the method comprising:
(a) contacting the biological sample with the isolated polypeptide of claim 7, such that SDF-1 and the isolated polypeptide form a complex; and
(b) isolating said complex to thereby isolate the SDF-1 from the biological sample.

37. A method of isolating SDF-1 from a biological sample, the method comprising:
(a) contacting the biological sample with the isolated polypeptide of claim 8, such that SDF-1 and the isolated polypeptide form a complex; and
(b) isolating said complex to thereby isolate the SDF-1 from the biological sample.

38. The isolated polynucleotide of claim 3, wherein the polypeptide is as set forth in SEQ ID NO: 2, 4 or 6.

39. The isolated polynucleotide of claim 4, wherein the polypeptide is as set forth in SEQ ID NO: 2, 4 or 6.

40. The isolated polypeptide of claim 5, as set forth in SEQ ID NO: 2, 4 or 6.

41. The isolated polypeptide of claim 6, as set forth in SEQ ID NO: 2, 4 or 6.

42. The method of claim 10, wherein the polypeptide is as set forth in SEQ ID NO: 2, 4 or 6.

43. The method of claim 33, wherein the polypeptide is as set forth in SEQ ID NO: 2, 4 or 6.

44. The isolated polynucleotide, of claim 3, wherein the polypeptide further comprises a heterologous amino acid sequence conjugated to said amino acid sequence.

45. The isolated polynucleotide, of claim 4, wherein the polypeptide further comprises a heterologous amino acid sequence conjugated to said amino acid sequence.

46. The isolated polypeptide of claim 5, wherein the polypeptide further comprises a heterologous amino acid sequence conjugated to said amino acid sequence.

47. The isolated polypeptide of claim 6, wherein the polypeptide further comprises a heterologous amino acid sequence conjugated to said amino acid sequence.

48. The method of claim 10, wherein the polypeptide further comprises a heterologous amino acid sequence conjugated to said amino acid sequence.

49. The method of claim 33, wherein the polypeptide further comprises a heterologous amino acid sequence conjugated to said amino acid sequence.

50. The isolated polynucleotide of claim 44, wherein said heterologous amino acid sequence comprises an immunoglobulin amino acid sequence.

51. The isolated polynucleotide of claim 45, wherein said heterologous amino acid sequence comprises an immunoglobulin amino acid sequence.

52. The isolated polypeptide of claim 46, wherein said heterologous amino acid sequence comprises an immunoglobulin amino acid sequence.

53. The isolated polynucleotide of claim 47, wherein said heterologous amino acid sequence comprises an immunoglobulin amino acid sequence.

54. The method of claim 48, wherein said heterologous amino acid sequence comprises an immunoglobulin amino acid sequence.

55. The method of claim 49, wherein said heterologous amino acid sequence comprises an immunoglobulin amino acid sequence.

56. The method of claim 33, wherein said CXCR4 associated medical condition is cancer or cancer metastasis.

57. The method of claim 33, wherein said CXCR4 associated medical condition is an inflammatory disease.

58. The method of claim 33, wherein said CXCR4 associated medical condition is AIDS.

59. The method of claim 33, wherein said CXCR4 associated medical condition is selected from the group consisting of idiopathic inflammatory myopathies (IIM), viral hepatitis, liver cirrhosis, primary biliary cirrhosis, primary sclerosing cholangitis, autoimmune hepatitis, cancer, cancer metastasis, small cell lung cancer (SCLC), osteosarcoma, inflammatory breast carcinoma (IBC), giant cell tumor (GCT) of bone, acute myeloblastic leukemia (AML), prostate cancer, multiple sclerosis, monocytic leukemia, arthritis, Atopic keratoconjunctivitis (AKC), diabetes and diabetic retinopathy.

60. The method of claim 34, wherein said CXCR4 associated medical condition is cancer or cancer metastasis.

61. The method of claim 34, wherein said CXCR4 associated medical condition is an inflammatory disease.

62. The method of claim 34, wherein said CXCR4 associated medical condition is AIDS.

63. The method of claim 34, wherein said CXCR4 associated medical condition is selected from the group consisting of idiopathic inflammatory myopathies (IIM), viral hepatitis, liver cirrhosis, primary biliary cirrhosis, primary sclerosing cholangitis, autoimmune hepatitis, cancer, cancer metastasis, small cell lung cancer (SCLC), osteosarcoma, inflammatory breast carcinoma (IBC), giant cell tumor (GCT) of bone, acute myeloblastic leukemia (AML), prostate cancer, multiple sclerosis, monocytic leukemia, arthritis, Atopic keratoconjunctivitis (AKC), diabetes and diabetic retinopathy.

64. The method of claim 35, wherein said CXCR4 associated medical condition is cancer or cancer metastasis.

65. The method of claim 35, wherein said CXCR4 associated medical condition is an inflammatory disease.

66. The method of claim 35, wherein said CXCR4 associated medical condition is AIDS.

67. The method of claim 35, wherein said CXCR4 associated medical condition is selected from the group consisting of idiopathic inflammatory myopathies (IIM), viral hepatitis, liver cirrhosis, primary biliary cirrhosis, primary sclerosing cholangitis, autoimmune hepatitis, cancer, cancer metastasis, small cell lung cancer (SCLC), osteosarcoma, inflammatory breast carcinoma (IBC), giant cell tumor (GCT) of bone, acute myeloblastic leukemia (AML), prostate cancer, multiple sclerosis, monocytic leukemia, arthritis, Atopic keratoconjunctivitis (AKC), diabetes and diabetic retinopathy.


SEQUENCE LISTING (14 sequences)

SEQ ID NO: 1 - DNA, 117 bp, Homo sapiens
  misc_feature: Polynucleotide coding for N-ter domain of CXCR4
atggaggggatcagtatatacacttcagataactacaccgaggaaatgggctcaggggactatgactccatgaaggaaccctgtttccgtgaagaaaatgctaatttcaataaaatc

SEQ ID NO: 2 - protein, 39 aa, Homo sapiens
  misc_feature: N-ter domain of CXCR4
MEGISIYTSDNYTEEMGSGDYDSMKEPCFREENANFNKI

SEQ ID NO: 3 - DNA, 822 bp, Artificial sequence
  A chimeric fusion comprised of CXCR4 peptide coding sequence fused to an immunoglobulin cDNA
atggaggggatcagtatatacacttcagataactacaccgaggaaatgggctcaggggactatgactccatgaaggaaccctgtttccgtgaagaaaatgctaatttcaataaaatcctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcgactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccaccgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggacctgcacaaccactacacgcagaagagcctctccctgtacccgggtaaagggccc

SEQ ID NO: 4 - protein, 274 aa, Artificial sequence
  A chimeric fusion comprised of CXCR4 peptide fused to an immunoglobulin
MEGISIYTSDNYTEEMGSGDYDSMKEPCFREENANFNKILEPKSCDKTHTCPPCPAPELLGGPSVFLFPPKPKDTLMISRTPEVTCVVVDVSHEDPEVKFDWYVDGVEVHNAKTKPREEQYNSTYRVVSVLTVLHQDWLNGKEYKCKVSNKALPAPTEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEDLHNHYTQKSLSLYPGKGP

SEQ ID NO: 5 - DNA, 93 bp, Homo sapiens
  misc_feature: Polynucleotide coding for ecl-2 domain of CXCR4
gccaacgtcagtgaggcagatgacagatatatctgtgaccgcttctaccccaatgacttgtgggtggttgtgttccagtttcagcacatcatg

SEQ ID NO: 6 - protein, 31 aa, Homo sapiens
  misc_feature: ecl-2 domain of CXCR4
ANVSEADDRYICDRFYPNDLWVVVFQFQHIM

SEQ ID NO: 7 - protein, 14 aa, Homo sapiens
  misc_feature: ecl-1 domain of CXCR4
DAVANWYFGNFLCK

SEQ ID NO: 8 - protein, 20 aa, Homo sapiens
  misc_feature: ecl-3 domain of CXCR4
SFILLEIIKQGCEFENTVHK

SEQ ID NO: 9 - DNA, 705 bp, Homo sapiens
  misc_feature: Ig coding sequence
ctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcgactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccaccgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggacctgcacaaccactacacgcagaagagcctctccctgtacccgggtaaagggccc

SEQ ID NO: 10 - protein, 104 aa, Homo sapiens
  misc_feature: Ig polypeptide
EPKSCDKTHTCPPCPAPELLGGPSVFLFPPKPKDTLMISRTPEVTCVVVDVSHEDPEVKFDWYVDGVEVHNAKTKPREEQYNSTYRVVSVLTVLHQDWLNGKEY

SEQ ID NO: 11 - DNA, 29 bp, Homo sapiens
cccaagcttatggaggggatcagtatata

SEQ ID NO: 12 - DNA, 28 bp, Homo sapiens
ccgctcgaggattttattgaaattagca

SEQ ID NO: 13 - DNA, 1691 bp, Homo sapiens
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 14 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS